(19) United States
(12) Patent Application Publication
(10) Pub. No.: US 2024/0253022 A1
(43) Pub. Date: Aug. 1, 2024

(54) HYDROXYAPATITE CATALYSTS FOR ISOBUTANOL SYNTHESIS

(71) Applicants: UOP LLC, Des Plaines, IL (US); China Petroleum & Chemical Corporation, Beijing (CN)

(72) Inventors: Richard LONG, Katy, TX (US); Tian RUAN, Katy, TX (US); Desmond SCHIPPER, Katy, TX (US); Vesna HAVRAN MUELLER, Houston, TX (US)

(21) Appl. No.: 18/565,619

(22) PCT Filed: Jun. 4, 2021
(86) PCT No.: PCT/US2021/035964

§ 371 Date: Nov. 30, 2023

(51) Int. Cl. B01J 27/185; B01J 27/187; C07C 29/34

(52) U.S. Cl. CPC B01J 27/1856; B01J 27/1853; B01J 27/187; C07C 29/34

(57) ABSTRACT

Metal-doped hydroxyapatite catalysts for isobutanol and propanol synthesis have been developed which exhibit good isobutanol yield in propanol-methanol and ethanol-methanol reactions. The metal-doped hydroxyapatites include, but are not limited to, one or more of metal-doped MgₓPO_{y}, CaₓPO_{y}, SrₓPO_{y} and BaₓPO_{y}. The metal-doped hydroxyapatites may have different phosphorus to alkaline earth ratios. Methods for making isobutanol and propanol using the metal-doped hydroxyapatite catalysts are also provided.

### BACKGROUND

Isobutanol is an organic solvent and a feedstock in the manufacturing of isobutyl acetate and isobutyl esters. It can also be blended directly with gasoline to improve octane number and combustion efficiency or be used as a neat alternative fuel. Isobutanol has relatively higher energy density and lower volatility compared to ethanol. In addition, it does not readily absorb water from air, preventing or reducing the corrosion of engines and pipelines. Although isobutanol has many potential uses, its synthesis is limited. Isobutanol is currently produced through the carbonylation of propylene. This process involves reacting propylene with carbon monoxide and hydrogen to generate butyraldehyde and isobutyraldehyde, hydrogenating them to n-butanol and isobutanol, followed by separation of the butanols. A new alternative technology is biomass fermentation. However, isobutanol selectivities in these two homogeneous processes are low, and the productivities are limited, resulting in a high cost of isobutanol.

Guerbet reaction is an alternative process for isobutanol synthesis from methanol and ethanol/propanol. This reaction is of special importance because it can produce value added isobutanol from the low-cost mixed alcohols. The Guerbet reaction takes place by a coupling process between alcohols on multi-functional catalysts with dehydrogenation activity, strong surface basicity, mild acidity, and hydrogenation activity. The reactions are below:

$${C}_{2} {H}_{5} O H + C {H}_{3} OH = {C}_{3} {H}_{7} OH + {H}_{2} O \qquad ( 1 )$$

$${C}_{3} {H}_{7} O H + C {H}_{3} O H = {C}_{4} {H}_{9} OH + {H}_{2} O \qquad ( 2 )$$

$${C}_{2} {H}_{5} O H + 2 C {H}_{3} O H = {C}_{4} {H}_{9} OH + 2 {H}_{2} O \qquad ( 3 )$$

As a result, various catalysts and processes for producing isobutanol from methanol, ethanol and propanol have been sought. For example, U.S. Pat. Nos. 5,581,602, 5,707,920, 5,770,541, 5,908,807, 5,939,352 and 6,034,141 describe precious metal loaded alkali metal doped ZnMnZr oxide catalysts for converting methanol and ethanol, or methanol, ethanol and propanol to isobutanol.

U.S. Pat. No. 5,559,275 discloses a process for the conversion of methanol, ethanol and propanol to higher branched oxygenates, such as isobutanol on a catalyst comprising a) a mixed oxide support having at least two components selected from Zn, Mg, Zr, Mn, Ti, Cr and La oxides; and b) an active metal selected from Pd, Pt, Ag, Rh, Co, and mixtures thereof.

Carlini, “Guerbet condensation of methanol with n-propanol to isobutyl alcohol over heterogeneous bifunctional catalysts based on Mg—Al mixed oxides partially substituted by different metal components,” Journal of Molecular Catalysis A: Chemical, 2005, 232, 13, describes Pd, Rh, Ni and Cu doped on Mg—Al mixed oxides for isobutanol synthesis from methanol and propanol.

Gabriëls, “Review of catalytic systems and thermodynamics for the Guerbet condensation reaction and challenges for biomass valorization,” Catalysis Science & Technology, 2015, 5, 3876, summarizes a series of catalysts for the reaction between methanol and ethanol/propanol, including alkali or alkaline earth supported on Al₂O₃, Ca or Sr hydroxyapatite, hydrotalcite, MgO, Mg(OH)₂, Rb—Li exchanged zeolite X and Na₂CO₃/NaX.

Therefore, there is a need for catalysts producing isobutanol and propanol from methanol, ethanol, and propanol, and for methods of using the catalysts.

### DETAILED DESCRIPTION

New metal-doped hydroxyapatite catalysts have been developed which exhibit good isobutanol yield and/or increased conversion of one or more of methanol, ethanol, and propanol in propanol-methanol and ethanol-methanol reactions. Methanol and ethanol can be reacted to form propanol, which can then react with methanol to form isobutanol using the metal-doped hydroxyapatite catalysts. Alternatively, methanol and propanol can be reacted directly using the metal-doped hydroxyapatite catalysts.

One aspect of the invention is a catalyst for isobutanol synthesis. In one embodiment, the catalyst comprises a metal-doped hydroxyapatite (HAP). Hydroxyapatite, also called hydroxylapatite, is a natural mineral form of calcium apatite with a formula Ca₅(PO₄)₃(OH), also written as Ca₁₀(PO₄)₆(OH)₂ to indicate that the crystal unit cell includes two entities. After calcination at high temperature, the material loses water to form Ca_{1.67}PO_{4.17}. The Ca in the HAP can be replaced partially or fully by other alkaline earth elements, such as Mg, Sr and Ba. Hence, the hydroxyapatites include, but are not limited to, one or more of MgₓPO_{y}, CaₓPO_{y}, SrₓPO_{y} and BaₓPO_{y}. The metal-doped hydroxyapatites may have different phosphorus to alkaline earth ratios, such as Mg_{1.67}PO_{4.17}, Ca_{1.67}PO_{4.17}, Sr_{1.67}PO_{4.17} and Ba_{1.67}PO_{4.17}. The value of x can be in the range of 1.5 to 3, or 1.5 to 2.1, and the value of y can be in the range of 4 to 5.5, or 4 to 4.6. In some embodiments, the hydroxyapatite comprises one or more of MgₓPO_{y}, SrₓPO_{y} and BaₓPO_{y}. They have various surface basic sites and acidic sites, which can contribute aldol condensation and dehydration of organic oxygenates.

The use of the metal-doped hydroxyapatite catalysts in isobutanol synthesis reactions resulted in significantly increased methanol conversion, ethanol conversion, and/or propanol conversion compared to hydroxyapatite catalysts without metal doping. Although not wishing to be bound by theory, the activity increase could be due to the enhancement in dehydrogenation activity and hydrogenation activity on these metal sites. Consequently, the productivities of isobutanol and propanol (for ethanol-methanol reactions) and/or conversion of one or more of methanol, ethanol, and propanol increased significantly, especially for Cu and Ir doped catalysts.

The hydroxyapatites are doped with one or more metals. The role of the metal is to improve hydrogenation and dehydrogenation activities. The metals comprise metals from Groups 7-11 of the Periodic Table. Suitable metals include, but are not limited to, Fe, Co, Ni, Cu, Ru, Rh, Pd, Ag, Re, Ir, Pt, Au, and combinations thereof. In some embodiments, when the hydroxyapatite is CaₓPO_{y}, only a single metal is used. The metal loading is in the range of 0.01 wt % to 50 wt %, or 0.1 wt % to 30 wt %, or 1 wt % to 20 wt %.

The catalysts may also contain alkali or alkaline earth metal oxides and/or salts as promoters to further improve the performance. The promoters comprise oxides and salts of alkali or alkaline earth metals from Groups 1 and 2 of the Periodic Table. Suitable promoters include, but are not limited to, oxides and/or salts of Li, Na, K, Rb, Cs, and combinations thereof. The salts could include, but are not limited to, carbonates, formates, acetates, nitrates, and combinations thereof. The role of the alkali or alkaline earth metal oxides/salts is to reduce ether formation through decreasing surface acidity. The loading of alkali or alkaline earth metal oxides/salts is in the range of 0.01 wt % to 15 wt %, or 0.1 wt % to 5 wt %, or 0.5 wt % to 3 wt %.

In one embodiment of the catalyst, the hydroxyapatite is CaₓPO_{y}, x is in a range of 1.5 to 3, and y is in a range of 4 to 5.5, and the metal is Cu or Ir.

Another aspect of the invention is method of making isobutanol. In one embodiment, the method comprises: reacting methanol and at least one of ethanol and propanol in the presence of a catalyst comprising a metal-doped hydroxyapatite, wherein the metal is selected from elements of Groups 7-11 of the Periodic Table.

The hydroxyapatites include, but are not limited to, one or more of MgₓPO_{y}, CaₓPO_{y}, SrₓPO_{y} and BaₓPO_{y}, as described above. The value of x can be in the range of 1.5 to 3, and the value of y can be in the range of 4 to 5.5. In some embodiments, the hydroxyapatite comprises one or more of MgₓPO_{y}, SrₓPO_{y} and BaₓPO_{y}.

The hydroxyapatites are doped with one or more metals, as described above. The metals comprise metals from Groups 7-11 of the Periodic Table. Suitable metals include, but are not limited to, Fe, Co, Ni, Cu, Ru, Rh, Pd, Ag, Re, Ir, Pt, Au, and combinations thereof. In some embodiments, when the hydroxyapatite is CaₓPO_{y}, there is a single metal. The metal loadings are described above.

The catalysts may also contain alkali or alkaline earth metal oxides and/or salts as promoters, as described above. The promoters comprise oxides and salts of metals from Groups 1 and 2 of the Periodic Table. Suitable promoters include, but are not limited to, Li, Na, K, Rb and Cs. The loading of the alkali or alkaline earth metal oxides/salts is described above.

In some embodiments, the hydroxyapatite is CaₓPO_{y}, x is in a range of 1.5 to 3, and y is in a range of 4 to 5.5, and the metal is Cu.

In some embodiments, the reaction takes place at a temperature in a range of about 150° C. to about 500° C., or about 200° C. to about 400° C., or about 250° C. to about 350° C.

In some embodiments, the reaction takes place at a pressure in a range of about 0.1 to about 200 atm, or about 1 to about 100 atm, or about 1 to about 50 atm.

In some embodiments, the reaction takes place at a methanol to ethanol molar ratio in a range of about 1:1 to about 20:1, or about 1:1 to about 10:1, or about 1:1 to about 4:1.

In some embodiments, the reaction takes place at a methanol to propanol molar ratio in a range of about 1:1 to about 20:1, or about 1:1 to about 5:1, or about 1:1 to about 2:1.

In some embodiments, the ethanol conversion is about 25% or more, or about 50% or more, or about 80% or more, or the propanol conversion is about 25% or more, or about 50% or more, or about 80% or more, or both.

Hydroxyapatites are generally prepared by a co-precipitation method, as described in the Examples. Next, metal salts are impregnated on the hydroxyapatites by incipient wetness impregnation.

### EXAMPLES

### Example 1: Ca_{1.67}PO_{4.17} (Reference)

Ca_{1.67}PO_{4.17} catalyst was prepared with co-precipitation. 63.3 g Ca(NO₃)₂·4H₂O was dissolved in 200 g deionized water in a beaker, and the pH value of the solution was adjusted to 11.0 with 25% tetramethylammonium hydroxide solution. A tetramethylammonium hydroxide solution with pH 11.0 was added to obtain 350 g of the solution.

In a second beaker, 21.23 g (NH₄)₂HPO₄ was dissolved in 200 g deionized water. Similarly, the pH value of the solution was adjusted to 11.0 with 25% tetramethylammonium hydroxide solution, and a tetramethylammonium hydroxide solution with pH 11.0 was added to get 350 g solution.

Subsequently, the calcium solution was added to the phosphorus solution with stirring. The mixture was stirred for additional 30 minutes at room temperature before it was heated to 80° C. and maintained at 80° C. for 3 hours. The slurry was cooled to room temperature, filtered with centrifuging, dried at 120° C. for 12 hours and calcined at 600° C. for 2 hours in air. Finally, the solid was crushed and sieved to 30-70 mesh prior to use.

### Example 2: 5% Cu/Ca_{1.67}PO_{4.17}

5% Cu/Ca_{1.67}PO_{4.17} was prepared with incipient wetness impregnation. 0.586 g Cu(NO₃)₂·2.5H₂O was dissolved in 1.5 g deionized water and then impregnated on 3.0 g 30-70 mesh Ca_{1.67}PO_{4.17} (Example 1). Subsequently, the solid was dried at 120° C. for 4 hours in air and then calcined at 400° C. for 4 hours.

### Example 3: 5% Pd/Ca_{1.67}PO_{4.17}

5% Pd/Ca_{1.6}PO_{4.17} was prepared with incipient wetness impregnation. 0.399 g Pd(NH₃)₄(HCO₃)₂ was dissolved in 1.35 g deionized water and then impregnated on 2.7 g 30-70 mesh Ca_{1.67}PO_{4.17} (Example 1). Subsequently, the solid was dried at 120° C. for 4 hours in air and then calcined at 400° C. for 4 hours.

### Example 4: 5% Pt/Ca_{1.67}PO_{4.17}

5% Pt/Ca_{1.67}PO_{4.17} was prepared with incipient wetness impregnation. 0.379 g H₂PtCl₆·6H₂O was dissolved in 1.35 g deionized water and then impregnated on 2.7 g 30-70 mesh Ca_{1.67}PO_{4.17} (Example 1). Subsequently, the solid was dried at 120° C. for 4 hours in air and then calcined at 400° C. for 4 hours.

### Example 5: 5% Ir/Ca_{1.67}PO_{4.17}

5% Ir/Ca_{1.67}PO_{4.17} was prepared with incipient wetness impregnation. 0.273 g IrCl₄ was dissolved in 1.55 g deionized water and then impregnated on 3.0 g 30-70 mesh Ca_{1.67}PO_{4.17} (Example 1). Subsequently, the solid was dried at 120° C. for 4 hours in air and then calcined at 300° C. for 4 hours.

### Example 6: 5% Cu/Sr_{1.67}PO_{4.17}

Sr_{1.67}PO_{4.17} catalyst was prepared with co-precipitation. 39.94 g Sr(NO₃)₂ was dissolved in 200 g deionized water in a beaker, and the pH value of the solution was adjusted to 11.0 with 25% tetramethylammonium hydroxide solution. A tetramethylammonium hydroxide solution with pH 11.0 was added to obtain 350 g of the solution.

In a second beaker, 14.95 g (NH₄)₂HPO₄ was dissolved in 200 g deionized water. Similarly, the pH value of the solution was adjusted to 11.0 with 25% tetramethylammonium hydroxide solution, and a tetramethylammonium hydroxide solution with pH 11.0 was added to get 350 g solution.

Subsequently, the strontium solution was added to the phosphorus solution with stirring. The mixture was stirred for additional 30 minutes at room temperature before it was heated to 80° C. and maintained at 80° C. for 3 hours. The slurry was cooled to room temperature, filtered with centrifuging, dried at 120° C. for 12 hours, and calcined at 600° C. for 2 hours in air. Finally, the solid was crushed and sieved to 30-70 mesh.

5% Cu/Sr_{1.67}PO_{4.17} was prepared with incipient wetness impregnation. 0.976 g Cu(NO₃)₂·2.5H₂O was dissolved in 1.3 g deionized water and then impregnated on 5.0 g 30-70 mesh Sr_{1.67}PO_{4.17}. Subsequently, the solid was dried at 120° C. for 4 hours in air and then calcined at 400° C. for 4 hours.

### Example 7

The Ca_{1.67}PO_{4.17} catalyst from Example 1 was tested in an autoclave for propanol-methanol reaction. 1.0 g catalyst and 33 g solution with 5:1 methanol/propanol molar ratio were loaded and tested at 350° C. for 10 hours. 42% propanol conversion and 21% methanol conversion were obtained while isobutanol productivity was 167 g/kg-h (Table 1).

### Example 8

The 5% Cu/Ca_{1.67}PO_{4.17} catalyst from Example 2 was tested in an autoclave for propanol-methanol reaction. 1.0 g catalyst and 33 g solution with 5:1 methanol/propanol molar ratio were loaded and tested at 350° C. for 10 hours. 81% propanol conversion and 72% methanol conversion were obtained while isobutanol productivity was 313 g/kg-h (Table 1).

### Example 9

The 5% Pd/Ca_{1.67}PO_{4.17} catalyst from Example 3 was tested in an autoclave for propanol-methanol reaction. 1.0 g catalyst and 33 g solution with 5:1 methanol/propanol molar ratio were loaded and tested at 350° C. for 10 hours. 67% propanol conversion and 69% methanol conversion were obtained while isobutanol productivity was 121 g/kg-h (Table 1).

### Example 10

The 5% Pt/Ca_{1.67}PO_{4.17} catalyst from Example 4 was tested in an autoclave for propanol-methanol reaction. 1.0 g catalyst and 33 g solution with 5:1 methanol/propanol molar ratio were loaded and tested at 350° C. for 10 hours. 80% propanol conversion and 75% methanol conversion were obtained while isobutanol productivity was 176 g/kg-h (Table 1).

### Example 11

The 5% Ir/Ca_{1.67}PO_{4.17} catalyst from Example 5 was tested in an autoclave for propanol-methanol reaction. 1.0 g catalyst and 33 g solution with 5:1 methanol/propanol molar ratio were loaded and tested at 350° C. for 10 hours. 77% propanol conversion and 47% methanol conversion were obtained while isobutanol productivity was 396 g/kg-h (Table 1).

### Example 12

The 5% Cu/Sr_{1.67}PO_{4.17} catalyst from Example 6 was tested in an autoclave for propanol-methanol reaction. 1.0 g catalyst and 33 g solution with 5:1 methanol/propanol molar ratio were loaded and tested at 350° C. for 10 hours. 72% propanol conversion and 69% methanol conversion were obtained while isobutanol productivity was 237 g/kg-h (Table 1).

### Example 13

The 5% Cu/Ca_{1.67}PO_{4.17} catalyst from Example 2 was tested in an autoclave for ethanol-methanol reaction. 1.0 g catalyst and 33 g solution with 6:1 methanol/ethanol molar ratio were loaded and tested at 350° C. for 10 hours. 78% ethanol conversion and 74% methanol conversion were obtained. The yields of propanol, isobutanol and n-butanol were 64, 66 and 13 g/kg-h, respectively (Table 2).

### Example 14

The 5% Cu/Sr_{1.67}PO_{4.17} catalyst from Example 6 was tested in an autoclave for ethanol-methanol reaction. 1.0 g catalyst and 33 g solution with 6:1 methanol/ethanol molar ratio were loaded and tested at 350° C. for 10 hours. 69% ethanol conversion and 66% methanol conversion were obtained. The yields of propanol, isobutanol and n-butanol were 77, 54 and 14 g/kg-h, respectively (Table 2).

### Example 15

The 5% Cu/Ca_{1.67}PO_{4.17} catalyst from Example 2 was tested in a fixed-bed reactor under the conditions of 331° C., 1000 psi, 8% propanol, 41% methanol, balance N₂, GHSV=4,000 ml/g-h. 83% propanol conversion and 17% methanol conversion were obtained. Isobutanol productivity reached 597 g/kg-h under the testing conditions (Table 3).

TABLE 1
Propanol-methanol reaction results in autoclave testing

| | Conversion | | Isobutanol |
|---|---|---|---|
| Example | Methanol | Propanol | productivity (g/kg-h) |
| Example 7 | 21% | 42% | 167 |
| Example 8 | 72% | 81% | 313 |
| Example 9 | 69% | 67% | 121 |
| Example 10 | 75% | 80% | 176 |
| Example 11 | 47% | 77% | 396 |
| Example 12 | 69% | 72% | 237 |

TABLE 2
Ethanol-methanol reaction results in autoclave testing

| | Conversion | | Productivity (g/kg-h) | | |
|---|---|---|---|---|---|
| Example | Methanol | Ethanol | Propanol | i-Butanol | n-Butanol |
| Example 13 | 74% | 78% | 64 | 66 | 13 |
| Example 14 | 66% | 69% | 77 | 54 | 14 |

TABLE 3
Propanol-methanol reaction results in fixed-bed reactor testing

| | Conversion | | Isobutanol |
|---|---|---|---|
| Example | Methanol | Propanol | productivity (g/kg-h) |
| Example 15 | 17% | 83% | 597 |

These results indicated that methanol conversion and propanol or ethanol conversion increased significantly in the isobutanol synthesis reactions after the transition metal and precious metals were added to the hydroxyapatite catalyst. Consequently, the production of isobutanol and propanol (for the ethanol-methanol reaction) increased significantly, especially for Cu and Ir doped catalysts.

The term “about” means within 10% of the value, or within 5%, or within 1%.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A catalyst for isobutanol and propanol synthesis comprising:
a metal-doped hydroxyapatite, wherein the metal is selected from elements of Groups 7-11 of the Periodic Table.

2. The catalyst of claim 1 wherein the hydroxyapatite comprises one or more of MgₓPO_{y}, CaₓPO_{y}, SrₓPO_{y} and BaₓPO_{y}, wherein x is in a range of 1.5 to 3, and y is in a range of 4 to 5.5.

3. The catalyst of claim 2 wherein x is in a range of 1.5 to 2.1, and y is in a range of 4 to 4.6.

4. The catalyst of claim 1, wherein the hydroxyapatite comprises one or more of MgₓPO_{y}, SrₓPO_{y} and BaₓPO_{y}, wherein x is in a range of 1.5 to 3, and y is in a range of 4 to 5.5.

5. The catalyst of claim 1, wherein the metal is selected from Fe, Co, Ni, Cu, Ru, Rh, Pd, Ag, Re, Ir, Pt, Au, and combinations thereof.

6. The catalyst of claim 5 wherein the metal is present in an amount of 0.01 wt % to 50 wt %.

7. The catalyst of claim 1, further comprising a promoter selected from oxides and/or salts of metals of Groups 1 and 2 of the Periodic Table.

8. The catalyst of claim 7 wherein the promoter is selected from Li, Na, K, Rb, Cs, and combinations thereof.

9. The catalyst of claim 7 wherein the promoter is present in an amount of 0.01 wt % to 15 wt %.

10. The catalyst of claim 1, wherein the hydroxyapatite is CaₓPO_{y}, wherein x is in a range of 1.5 to 3, and y is in a range of 4 to 5.5, and wherein the metal is Cu or Ir.

11. A method of making isobutanol and propanol comprising:
reacting methanol and at least one of ethanol and propanol in the presence of a catalyst comprising a metal-doped hydroxyapatite, wherein the metal is selected from elements of Groups 7-11 of the Periodic Table.

12. The method of claim 11 wherein the hydroxyapatite comprises one or more of MgₓPO_{y}, CaₓPO_{y}, SrₓPO_{y} and BaₓPO_{y}, wherein x is in a range of 1.5 to 3, and y is in a range of 4 to 5.5.

13. The method of claim 11, wherein the hydroxyapatite comprises one or more of MgₓPO_{y}, SrₓPO_{y} and BaₓPO_{y}, wherein x is in a range of 1.5 to 3, and y is in a range of 4 to 5.5.

14. The method of claim 11, wherein the metal is selected from Fe, Co, Ni, Cu, Ru, Rh, Pd, Ag, Re, Ir, Pt, Au, and combinations thereof.

15. The method of claim 11 further comprising a promoter selected from oxides and salts of metals of Groups 1 and 2 of the Periodic Table.

16. The method of claim 15 wherein the promoter is selected from Li, Na, K, Rb, and Cs.

17. The method of claim 11, wherein the hydroxyapatite is CaₓPO_{y}, wherein x is in a range of 1.5 to 3, and y is in a range of 4 to 5.5, and wherein the metal is Cu or Ir.

18. The method of claim 11, wherein the reaction takes place at a temperature in a range of about 150° C. to about 500° C., or at a pressure in a range of about 0.1 to about 200 atm, or both.

19. The method of claim 11, wherein the reaction takes place at a methanol to ethanol molar ratio in a range of about 1:1 to about 20:1; or at a methanol to propanol molar ratio in a range of about 1:1 to about 20:1.

20. The method of claim 11, wherein ethanol conversion is about 25% or more, or propanol conversion is about 25% or more, or both.
